(19) United States
(12) Patent Application Publication
(10) Pub. No.: US 2017/0049367 A1
(43) Pub. Date: Feb. 23, 2017

(54) Reusable Respiratory Effort Sensor Module

(71) Applicant: Dymedix Corporation, Shoreview, MN (US)

(72) Inventors: James P. Moore, Bloomington, MN (US); Alan B. Jones, Maple Grove, MN (US); Todd M. Eiken, Lindstrom, MN (US)

(21) Appl. No.: 14/827,676

(22) Filed: Aug. 17, 2015

(51) Int. Cl. A61B 5/113; A61B 5/00; A61B 5/08

(52) U.S. Cl. CPC A61B 5/1135; A61B 5/08; A61B 5/4806; A61B 5/6831

(57) ABSTRACT

A respiratory effort sensing belt that incorporates a PVDF transducer is constructed such that the transducer is contained within an elastic, moisture impervious plastic envelope and affixed to the envelope are a pair of connectors adapted to receive opposed ends of a body-encircling, single-use band and which provide the ability to adjust belt tension. Because of the construction of the sensor module, it may be sterilized for reuse.

### CROSS REFERENCE TO RELATED APPLICATIONS

This application is a continuation-in-part of application Ser. No. 14/339,778, filed Jul. 24, 2014, entitled REUSABLE RESPIRATORY EFFORT SENSOR MODULE, which is deemed incorporated herein by reference in its entirety.

### STATEMENT REGARDING FEDERALLY SPONSORED RESEARCH OR DEVELOPMENT

Not applicable

### BACKGROUND OF THE INVENTION

I. Field of the Invention

This invention relates generally to apparatus for monitoring respiratory activity in the course of a sleep study and more particularly to a respiratory effort sensor designed such that the sensor electronics and associated leads for connecting the electronics to a polysomnograph machine (PSG) or home sleep test (HST) device is adapted for connection to a body-encircling band and remains reusable and only the low-cost, body-encircling band position is disposable following a use thereof.

II. Discussion of the Prior Art

In published U.S. Application 2008/0275356, there is described a respiratory effort belt that has been marketed by Dymedix Corporation of Shoreview, Minn. for a number of years. It comprises a PVDF film member having first and second metalized major surfaces to which a pair of approximately six foot long tabbed leads is electrically connected. The resulting assembly is permanently affixed to the surface of a body-encircling belt member. Signals are generated when the PVDF film on the belt member is stressed due to stretching as the user breathes in and out. The leads connect to a PSG, allowing the signals to be used in diagnosis and analysis.

Approximately 95% of the manufacturing cost of the respiratory effort belt resides in its sensor electronics and associated leads while the remaining 5% of the cost resides in the body-encircling belt on which the sensor electronics are affixed.

For infection control reasons, many sleep labs dispose of the respiratory effort belts after a use on a given patient. It is, of course, advantageous to reduce the cost of the diagnostic procedure and the present invention achieves this desired result.

### SUMMARY OF THE INVENTION

In accordance with the present invention, there is provided a respiratory effort belt comprising a PVDF film strip having a layer of metallization on opposed major surfaces thereof. First and second insulated conductive leads, each with first and second ends, have the first end of the first lead electrically connected to the layer of metallization on one of the opposed major surfaces and the second lead electrically connected to the layer of metallization on another of the opposed major surfaces. A flexible, elastic, moisture impervious, generally rectangular envelope is made to contain the metalized PVDF film strip and only a distal end portion of the first and second conductive leads. A pair of connectors is releasably attached individually to the first and second ends of the envelope. A disposable elastic or inelastic band, whose length is sufficient to encircle a human's torso, is adapted to be selectively joined and released to and from the pair of connectors. This allows the band to be disposed of after use while the envelope, with its connectors and containing the sensor element and leads, remains reusable following sterilization.

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which like numerals in the several views refer to corresponding parts.

FIG. 1 is a partial frontal view of a respiratory effort belt comprising a preferred embodiment of the present invention;

FIG. 2 is an exploded view of the reusable sensor module;

FIG. 3 is a front plan view of an alternative connector attachable to a first end of a sensor module;

FIG. 4 is a side view of the connector of FIG. 3;

FIG. 5 is a front plan view of an alternative connector attachable to a second end of the sensor module;

FIG. 6 is a side view of the connector of FIG. 5;

FIG. 7 is a front view of a sensor module with the connectors of FIGS. 3 and 5 joined thereto;

FIG. 8 is a front view of a sensor module having two connectors like those of FIGS. 5 and 6; and

FIG. 9 is a view of a belt used with the sensor module of FIG. 7.

### DESCRIPTION OF THE PREFERRED EMBODIMENTS

This description of the preferred embodiments is intended to be read in connection with the accompanying drawings, which are to be considered part of the entire written description of this invention. In the description, relative terms such as “lower”, “upper”, “horizontal”, “vertical”, “above”, “below”, “up”, “down”, “top” and “bottom” as well as derivatives thereof (e.g., “horizontally”, “downwardly”, “upwardly”, etc.) should be construed to refer to the orientation as then described or as shown in the drawings under discussion. These relative terms are for convenience of description and do not require that the apparatus be constructed or operated in a particular orientation. Terms such as “connected”, “connecting”, “attached”, “attaching”, “join” and “joining” are used interchangeably and refer to one structure or surface being secured to another structure or surface or integrally fabricated in one piece, unless expressively described otherwise.

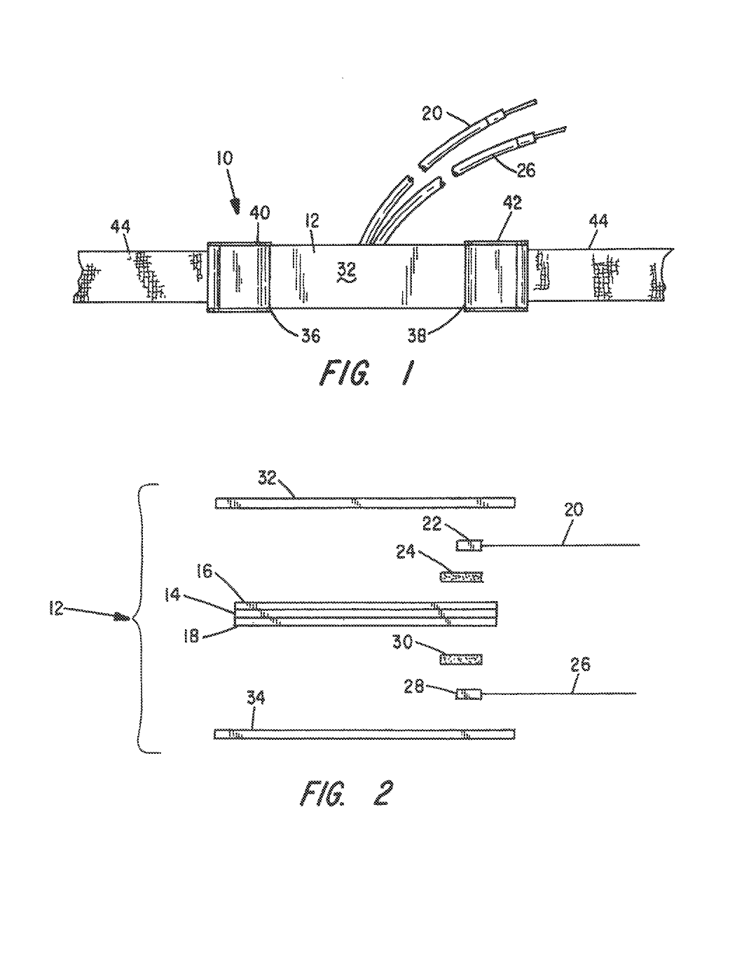

Referring first to FIG. 1, there is indicated generally by numeral 10 a respiratory effort belt constructed in accordance with the present invention. It is seen to comprise a reusable, sterilizable sensor module 12, the construction of which is best illustrated in the exploded view of FIG. 2. In FIG. 2, a strip of polyvinylidine fluoride (PVDF) is identified by numeral 14. The strip is generally rectangular and deposited on the opposed major surfaces thereof are metallization layers 16 and 18. An electrical lead 20 has a conductive terminal pad 22 bonded to the metallization layer 16 by a conductive adhesive member 24. Likewise, an electrical lead 26 has its conductive terminal pad 28 conductively affixed to the metallization layer 18 by means of a conductive adhesive layer 30.

The PVDF film 14, as well as distal end portions of the leads 20 and 26, is contained within a moisture impervious plastic envelope comprising elastic polymer layers 32 and 34 that are laminated together about the perimeters thereof to form the envelope.

Without limitation, the resulting module 12 may be about 5 inches in length and about 1½ inches in width.

Referring again to FIG. 1, affixed to the end portions 36 and 38 of the envelope comprising the module 12 are connectors 40 and 42 that are used to releasably couple the module 12 to a body-encircling band 44. Without limitation, the connectors 40 and 42 preferably comprise cam buckles, many types of which are commercially available. Such cam buckles releasably clamp to the opposed ends 36 and 38 of the envelope comprising the module 12 and permit adjustment of the body-encircling band 44 to a desired degree of snugness.

It is contemplated that the body-encircling band 44 be of an inexpensive cloth fabric that is somewhat elastic.

In use, the respiratory effort belt 10 will be placed about a patient's torso, either about the chest or abdomen, and any slack taken up by tensioning the band with the cam buckle 40 or 42 open and when the tension is deemed appropriate, the cam lever is released, locking the band 44 from moving relative to the module 12. As the subject breathes, the envelope comprising the sensor module 12, being elastic, will stretch upon inspiration and shrink during expiration. This places corresponding stresses on the PVDF film element 14 thereby producing, via the piezoelectric effect, electrical output signals on the leads 20 and 26.

Upon completion of a patient's sleep study, the body-encircling band 44 can be separated from the buckles 40 and 42 and properly disposed of. The module 12, however, can be retained for reuse following sterilization and/or cleaning and only the relatively inexpensive band 44 needs replacement. In that the great majority of the cost resides in the module 12 and because this module is reusable, a substantial savings results for the consumer who need only replace the fabric belt 44 and not the entire respiratory effort belt each time the belt is used.

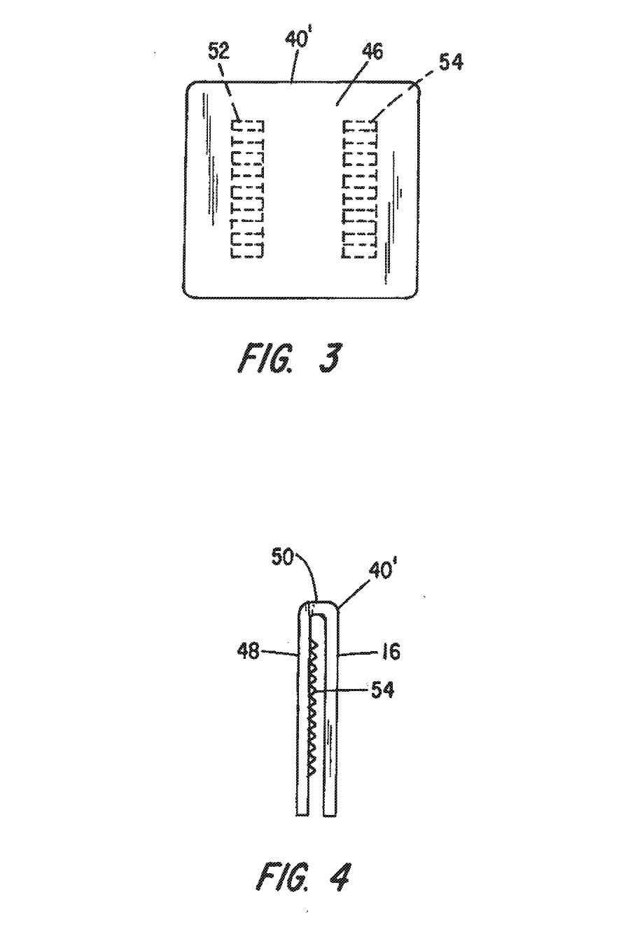

FIGS. 3 and 4 illustrate an alternative connector construction 40′ adapted to be attached to a first end of the sensor module 12. It is seen to comprise a molded plastic piece having generally planar front and rear surfaces 46 and 48 joined together at an upper end 50 to form a U-shaped cross section. Integrally molded on the inner side of the rear surface 48 are first and second rows of teeth 52 and 54. The upper end 50 is flexible such that front surface 46 can be squeezed against the rows of teeth 52 and 54 to engage the teeth with the belt fabric.

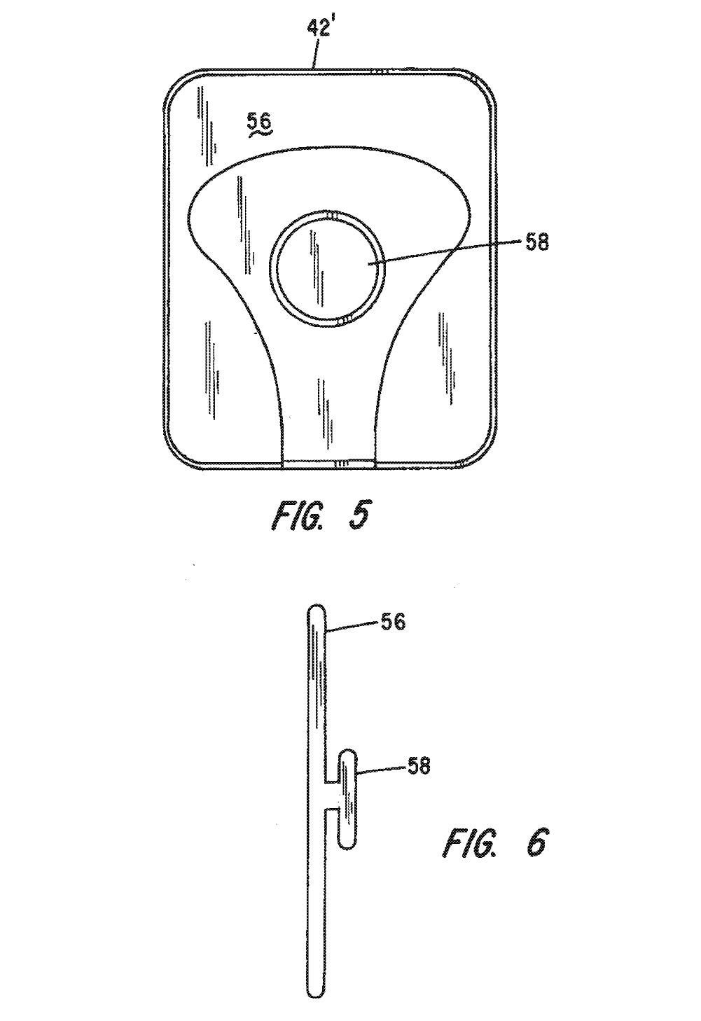

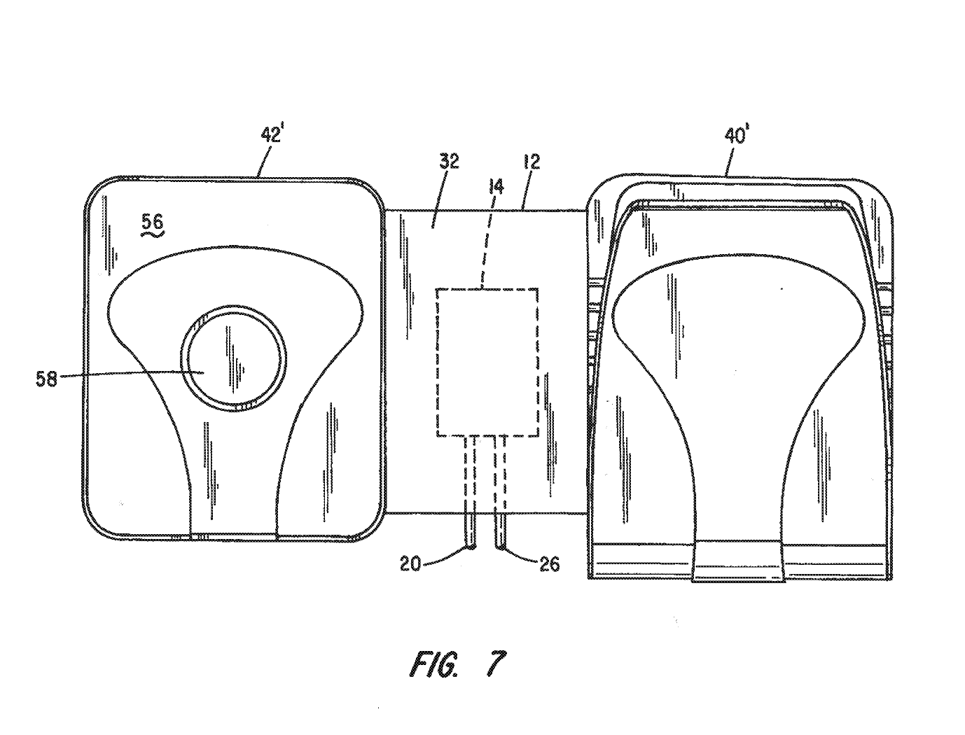

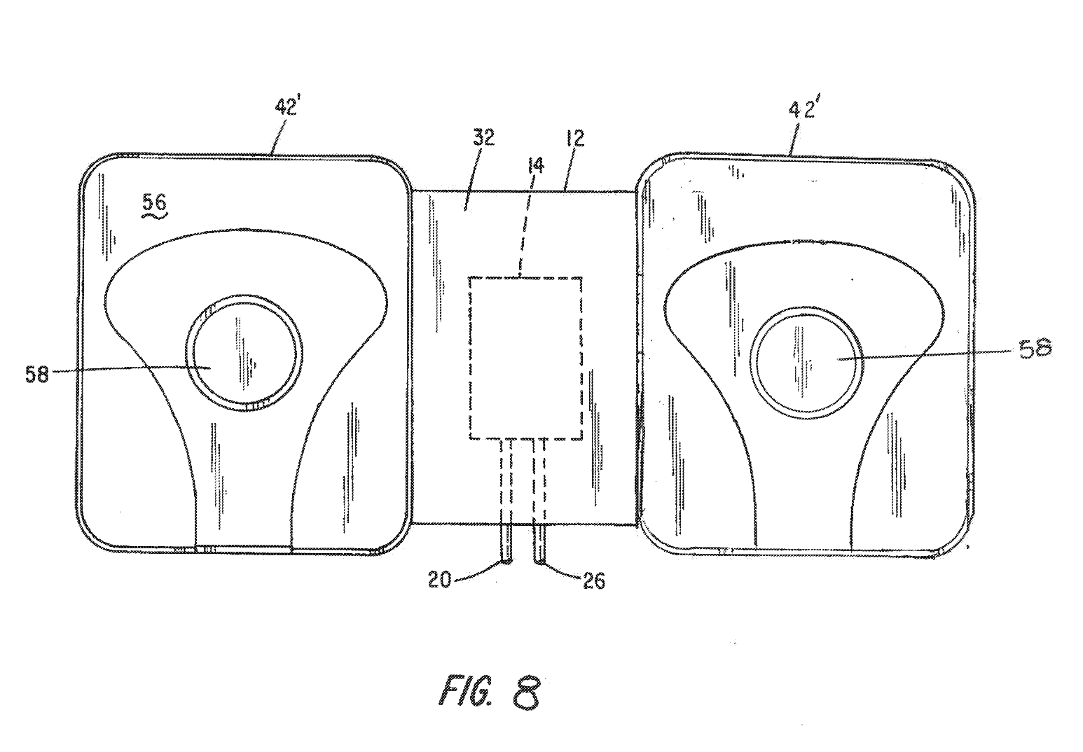

FIGS. 5 and 6 illustrate an alternative connector construction 42′ adapted to be attached to a second end of the sensor module 12. It is seen to comprise a molded, plastic, rectangular and generally planar sheet 56 and having an integrally molded and rounded button member 58 joined to a central portion of the sheet 56 and projecting outwardly therefrom. FIG. 7 shows the reusable PVDF sensor module 12 permanently joined to the connectors 40′ and 42′. More particularly, the envelope 32 containing the PVDF film 14 is adhesively or otherwise bonded to back surfaces of the connectors 40′ and 42′.

FIG. 8 illustrates yet another reusable sensor module embodiment which two of the connectors of FIGS. 5 and 6 are utilized. The moisture impervious envelope 32 containing the PVDF transducer 14 is permanently bonded to the connectors 42′, one on each end thereof.

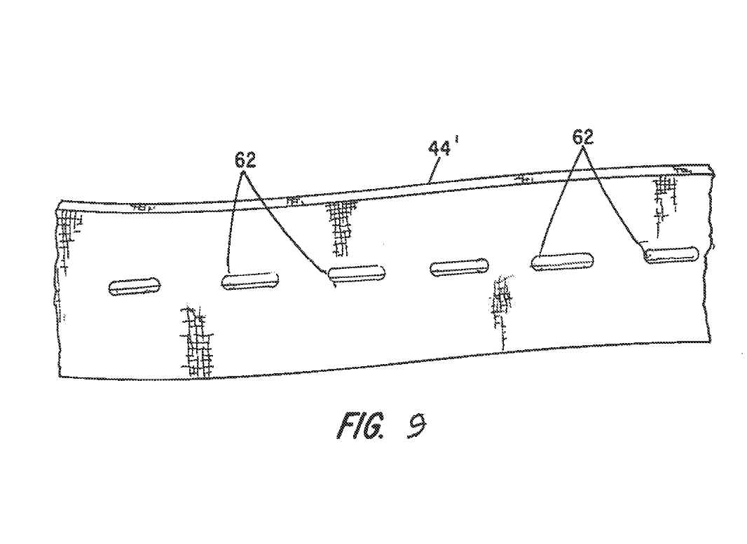

FIG. 9 illustrates the body encircling flexible, disposable, elastic fabric belt 44′. It is seen to include a series of regularly, longitudinally spaced buttonhole slits 62 formed through its thickness.

In use with the embodiment of FIG. 7, one end of the belt 44′ will be inserted into the gap of the U-shaped connector 40′ and by squeezing the surface 46 relative to surface 48, the rows of teeth 52, 54 will penetrate into the belt material and be releasably secured to one end thereof. The belt 44′ will then be deployed from a supply roll thereof and wrapped about the torso of the patient and when snug, the button member 58 of connector 42′ will be made to pass through a nearby one of the buttonholes 62 in the fabric belt 44 to secure the belt to the sensor module 12. The excess belt material still on the roll can now be cut free of the deployed sensor belt. Any further needed adjustment can be made by repositioning the belt on the teeth of the connector 40′.

With the embodiment of FIG. 8, a free end of the belt material of FIG. 9 is affixed to the button 58 of one of the connectors 42′ and as the belt material is dispensed from a roll thereof, it is wrapped above the subject and when snug, is fitted onto one button 58 of the other connector 42′ and then cut off from the roll of belt material.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices. Also, various modifications, both as to the equipment and operating procedures, can be accomplished without departing from the scope of the invention itself.

What is claimed is:

1. A respiratory effort belt comprising:
a) a PVDF film strip having a layer of metallization on opposed major surfaces thereof;
b) first and second insulated leads each with first and second ends, the first end of the first lead electrically connected to the layer of metallization on one of the opposed major surfaces and the second lead electrically connected to the layer of metallization on another of the opposed major surfaces;
c) a flexible, elastic, moisture impervious, generally rectangular elastomeric envelope containing the PVDF film strip and only a distal end portion of the first and second conductive leads, the envelope having first and second end portions; and
d) a pair of connectors attached individually to said first and second end portions of the envelope and adapted to be releasably affixed to opposed ends of a disposable band of a length sufficient to encircle a human's torso.

2. The respiratory effort belt of claim 1 wherein the connectors comprise cam buckles.

3. The respiratory effort belt of claim 1 wherein the band comprises a web fabric.

4. The respiratory effort belt of claim 3 wherein the web fabric includes a plurality of regularly longitudinally spaced and aligned apertures there through.

5. The respiratory effort belt of claim 1 wherein one of said pair of connectors comprises first and second planar surfaces joined to one another at corresponding edges thereof to define a gap there between, one of the planar surfaces including a plurality of teeth for releasably engaging the band when inserted in the gap.

6. The respiratory effort belt of claim 4 wherein at least one of the pair of connectors comprises a flat rectangular surface with a button member joined thereto in a central portion of the surface and projecting outwardly from the surface, the button member adapted to fit through a selected one of the holes in the web fabric.

7. The respiratory effort belt of claim 4 wherein each of the pair of connectors comprises a flat rectangular surface with a button member joined thereto in a central portion of the surface and projecting outwardly from the surface, the button member adapted to fit through a selected one of the holes in the web fabric.
